(19) United States
(12) Patent Application Publication
(10) Pub. No.: US 2024/0409492 A1
(43) Pub. Date: Dec. 12, 2024

(54) METHOD FOR CLEAVING (POLY)URETHANES

(71) Applicant: Covestro Deutschland AG, Leverkusen (DE)

(72) Inventors: Matthias Leven, Köln (DE); Norah Heinz, Leverkusen (DE); Jens Langanke, Mechernich (DE); Torsten Heinemann, Leichlingen (DE); Kai Laemmerhold, Odenthal (DE); Dirk Hinzmann, Pulheim (DE); Tugrul Nalbantoglu, Aachen (DE); Nicolas Vogt, Köln (DE); Walter Leitner, Aachen (DE); Elena Dirksen, Aachen (DE)

(21) Appl. No.: 18/699,687

(22) PCT Filed: Oct. 26, 2022
(86) PCT No.: PCT/EP2022/079864

§ 371 Date: Apr. 9, 2024

(30) Foreign Application Priority Data
Oct. 29, 2021 (EP) 21205590.9
Sep. 30, 2022 (EP) 22198948.6

(51) Int. Cl. C07C 209/62; C07C 29/09; C07C 29/128; C07C 29/74; C07C 209/84; C07C 209/86; C07C 269/04; C07C 269/08

(52) U.S. Cl. CPC C07C 209/62; C07C 29/095; C07C 29/1285; C07C 29/74; C07C 209/84; C07C 209/86; C07C 269/04; C07C 269/08

(57) ABSTRACT

The invention relates to a method for cleaving urethanes, in particular polyurethanes, by means of chemolysis (alcoholysis, hydrolysis, or hydroalcoholysis) in the presence of a catalyst. The chemolysis is characterized in that a salt of an oxyacid of an element of the fifth, fourteenth, or fifteenth group of the periodic table of elements or a mixture of two or more such salts is used as the catalyst, the pKb value of the anion of said salt of the oxyacid ranging from 0.10 to 6.00, preferably 0.25 to 5.00, particularly 0.50 to 4.50, wherein the catalyst does not comprise carbonate when the chemolysis is carried out as an alcoholysis (Ia), and the catalyst does not contain carbonate, orthophosphate, or metaphosphate when the chemolysis is carried out as a hydroalcoholysis.

The present invention relates to a method of cleaving urethanes, especially polyurethanes, by chemolysis (alcoholysis, hydrolysis or hydroalcoholysis) in the presence of a catalyst. It is a feature of chemolysis that the catalyst used is a salt of an oxoacid of an element from the fifth, fourteenth or fifteenth group of the Periodic Table of the Elements or a mixture of two or more such acids, where the pK_{B} of the anion of the salt of the oxoacid is in the range from 0.10 to 6.00, preferably 0.25 to 5.00, more preferably 0.50 to 4.50, and wherein the catalyst in the case of performance of the chemolysis as alcoholysis (Ia) does not comprise any carbonate and the catalyst in the case of performance of the chemolysis as hydroalcoholysis does not comprise any carbonate, any orthophosphate or any metaphosphate. The method of the invention enables the recovery of valuable raw materials from industrially produced urethanes, especially polyurethanes, after they have fulfilled their original purpose, and hence avoids loss of such raw materials as would arise, for instance, in the case of disposal by incineration or by landfill.

Urethanes are versatile products. Polyurethanes in particular enjoy a variety of applications in industry and in everyday life. In the case of the polyurethanes, distinctions are typically made between polyurethane foams and what are known as “CASE” products, with “CASE” being a collective term for polyurethane coatings (e.g., paints), adhesives, sealants and elastomers. The polyurethane foams are typically divided into rigid foams and flexible foams. Common to all of these products in spite of their heterogeneity is the basic polyurethane structure, which is formed by the polyaddition reaction of a polyfunctional isocyanate and of a polyol and which in the case, for example, of a polyurethane based on a diisocyanate O═C═N—R—N═C═O and a diol H—O—R′—O—H (where R and R′ denote organic radicals) can be represented as

˜˜˜[O—R′—O—(O═C)—HN—R—NH—(C═O)]˜˜˜.

Even simple (nonpolymeric) urethanes feature the urethane bond.

It is precisely the great economic success of the polyurethanes in particular that is responsible for the large amounts of polyurethane waste generated (for example from old mattresses or seating furniture) that must be sent to a sensible use. The mode of reuse that is the easiest to implement technically is that of incineration, with the heat of combustion released being utilized for other processes, examples being industrial processes. However, this does not allow raw material cycles to be completed. Another mode of reuse is called “physical recycling”, which sees polyurethane wastes mechanically comminuted and used in the production of new products. This type of recycling naturally has its limits and there has therefore been no lack of attempts to recover the basic raw materials of polyurethane production by retrocleavage of the polyurethane bonds (called “chemical recycling”). These raw materials to be recovered comprise primarily polyols (i.e., in the example above, H—O—R′—O—H). In addition, it is also possible through hydrolytic cleavage of the urethane bond to recover amines (i.e., in the example above, H₂N—R—NH₂) which can be phosgenated to afford isocyanates (in the aforementioned example to afford O═C═N—R—N═C═O) after workup.

A variety of chemical recycling approaches have been developed in the past. The three most important are briefly summarized as follows:
1. Hydrolysis of urethanes by reaction with water to recover amines and polyols with formation of carbon dioxide.
2. Glycolysis of urethanes by reaction with alcohols, with replacement of the polyols incorporated in the urethane groups by the alcohol used and hence release of the polyols. This process is commonly referred to in the literature as transesterification (more accurately: transurethanization). Regardless of the exact nature of the alcohol used, this mode of chemical recycling is dubbed glycolysis in the literature, a term that really applies only for glycol. In the context of the present invention the term alcoholysis is thus generally used. A glycolysis may be followed by a hydrolysis. If such a hydrolysis is effected with the unchanged glycolysis mixture, reference is made to a
3. Hydroglycolysis of urethane bonds by reaction with alcohols and water. It is of course possible for alcohol and water also both to be present from the very start of the chemolysis, in which case the above-described processes of glycolysis and hydrolysis proceed in parallel. In the context of the present invention, analogously with No. 2, the term hydroalcoholysis is used.

For these three approaches, in the context of the present invention, the collective term chemolysis is used.

A summary of the known methods of polyurethane recycling is offered by the review article by Simón, Borreguero, Lucas and Rodriguez in Waste Management 2018, 76, 147-171 [1].

International patent application WO 2022/171586 A1 describes the cleavage of polyurethanes using a carbonate, a hydrogencarbonate, an orthophosphate, a monohydrogen-orthophosphate, a metaphosphate or a mixture of two or more of the aforementioned metal salts as catalysts for a hydroalcoholysis (No. 3).

Only a few of the chemical recycling processes known from the literature are in sustained operation on an industrial scale; many have not even reached pilot scale [1]. In view of generally increased environmental awareness and increased efforts to configure industrial processes to be as sustainable as possible—both of which are fundamentally in favor of chemical recycling—this shows clearly that the chemical recycling of polyurethane products is still by no means mature from a technical and economic point of view. Challenges exist, for example, with regard to the efficiency of the catalytic urethane cleavage. Conventional method approaches that require reaction temperatures about 200° C. generally lead to significantly discolored products containing high proportions of by-products such as N-alkylated amines, which make it considerably more difficult to recycle recovered raw materials or can even make the recycling uneconomic.

The article Chemical degradation of polyurethanes, 3., Degradation of microporous polyurethane elastomer by diethyl phosphonate and tris(1-methyl-2-chloroethyl) phosphate by K. Troev et al. from Polymer Degradation and Stability 2000, 70, 43-48, describes a breakdown of a polyurethane elastomer based on diphenylmethane diisocyanate and a polyester polyol (Bayflex 2003E) with superstoichiometric amounts of diethyl phosphonate ((CH₃CH₂O)₂P(O)H, a diester of phosphonic acid) and tris(1-methyl-2-chloroethyl) phosphate ((CH₂ClCH(CH₃)O)₃P(O), a triester of phosphoric acid). This article is part of a series concerned with methods of polyurethane breakdown other than the known methods of hydrolysis (see No. 1 above), glycolysis (see No. 2 above) and aminolysis (not described above) (see the introduction to the article).

The reaction products formed were characterized chemically and include firstly the products 1 to 3, which can be regarded immediately as transesterification products of the polyester blocks of the polyurethane elastomer with the alkoxyphosphorus compounds (the “repeat unit” shown in the article does not fit with the other details in the article relating to the polyurethane elastomer; it can be assumed that the polyester blocks are much longer than shown). Secondly formed in addition are N-alkylated urethanes (in the form of salts with a positive charge on the urethane nitrogen—product 4).

What is referred to in the article as the “exchange reaction” between ethoxy groups or 1-methyl-2-chloroethoxy groups on the one hand and the urethane groups on the other hand is thus based at least predominantly, if not even entirely, on a cleavage of the ester bonds within the polyester blocks, i.e. is essentially not an exchange reaction of the polyurethane structure but of the polyester structure. In the case of diethyl phosphonate, products 1 to 3 are formed in a total of 30.6%, and the N-alkylated salt (product 4) at 51.6%. Such a product mixture, more than half of which, moreover, consists of N-alkylated compounds that cannot be hydrolyzed to amines and are therefore unavailable for re-phosgenation, is completely unusable for meaningful chemical recycling.

U.S. Pat. No. 4,159,972 describes a recycling method for flexible polyurethane foam, comprising (a) the dissolving of the foam in a diol of low molecular weight, (b) mixing-in a high molecular weight polyol suitable for production of flexible urethane foam, (c) removing the diol solvent of low molecular weight from the resultant mixture under reduced pressure, and (d) recovering the remaining polyol product. The polyol product thus recovered can be used in the production of new polyurethane foam. The foaming catalyst disclosed is dibutyl[bis(dodecanoyloxy)]-stannane.

EP 0 835 901 A2 describes a process for producing recycled polyols by glycolysis using a catalyst. Suitable catalysts described are Lewis acids (such as zinc chloride, iron chloride, aluminum chloride or mercury chloride), carboxylic acids (such as acetic acid, formic acid, propionic acid, butyric acid or benzoic acid), inorganic acetates (such as magnesium acetate, lead acetate, calcium acetate, potassium acetate, zinc acetate, sodium acetate or “phosphorus acetate”) and alkali metal salts (such as sodium carbonate, sodium hydrogencarbonate, calcium hydroxide, potassium hydroxide or sodium hydroxide).

The article “Polyurethane flexible foam recycling via glycolysis using Zn/Sn/Al hydrotalcites as heterogeneous catalyst” by Y. D. Morcillo-Bolanos et al. in Revista Facultad de Ingenieria, Universidad de Antioquia, 2018, 87, 77-85, is concerned with chemical polyol recovery from flexible polyurethane foam wastes by glycolysis with diethylene glycol under catalysis by Zn/Sn/Al hydrotalcite (HTC) as heterogeneous catalyst.

The article “Microwave-assisted Polyurethane Bond Cleavage via Hydroglycolysis Process at Atmospheric Pressure” by M. M Alavi Nikje et al. in Journal of Cellular Plastics, 2008, 44, 367-380 is concerned with the hydroglycolysis of flexible polyurethane foam under the influence of microwave radiation. This involves using mixtures of glycerol, water and sodium hydroxide.

The article “Methods of polyurethane and polyurethane composites recycling and recovery: A review” by K. M Zia et al. in Reactive & Functional Polymers, 2007, 67, 675-692 gives an overview of various methods of polyurethane recycling.

There was thus a need for further improvements in the field of chemical recycling of polyurethane products. In particular, it would be desirable to provide a method in which chemolysis is catalyzed efficiently and can therefore preferably be conducted at comparatively low temperature.

Taking account of this requirement, the present invention provides a method of cleaving urethanes (especially polyurethanes) by chemolysis (I), comprising (A) the providing of a urethane (especially polyurethane) based on an isocyanate component and an alcohol component, followed by (B) the chemolysis (I) of the urethane with a chemolysis reagent, wherein the chemolysis (I) is conducted with addition of a catalyst as one of the following conversions (in this regard, see the elucidations in the section on “Chemolysis methods (Ia), (Ib) and (Ic)” further down):
(Ia) alcoholysis of the (poly)urethane, where the chemolysis reagent is an alcohol,
(Ib) hydrolysis of the (poly)urethane, where the chemolysis reagent is water,
or
(Ic) hydroalcoholysis of the (poly)urethane, where the chemolysis reagent comprises alcohol and water, which are preferably added successively in that sequence,
   wherein the catalyst comprises a salt of an oxoacid of an element from the fifth, fourteenth or fifteenth group of the Periodic Table of the Elements or a mixture of two or more such acids, where the pK_{B} (determined according to the elucidations in the section on “Determination of the pK_{B} values” further down) of the anion of the salt of the oxoacid is in the range from 0.10 to 6.00, preferably 0.25 to 5.00, more preferably 0.50 to 4.50, and wherein the catalyst in the case of performance of the chemolysis as alcoholysis (Ia) does not comprise any carbonate and the catalyst in the case of performance of the chemolysis as hydroalcoholysis (Ic) does not comprise any carbonate, any orthophosphate or any metaphosphate.

Completely surprisingly, it has been found that the hydrolysis of the urethane bond is possible with the aid of anionic compounds having low to moderate pK_{B} values even on a catalytic scale without stoichiometric use thereof.

Urethanes in the context of the present invention are the addition products (occasionally also referred to, albeit not entirely correctly, as condensation products) that form by reaction of an isocyanate (=isocyanate component in the urethane preparation) with a mono- or polyol (=alcohol component in the urethane preparation). In the case of use of polyfunctional isocyanates and of polyols, the polyurethanes outlined above are formed. Polyurethanes in particular generally contain not only the (poly)urethane base structure outlined above but also other structures, for example structures having urea bonds. The presence of such structures diverging from the pure (poly)urethane base structure in addition to (poly)urethane structures does not depart from the scope of the present invention.

In the terminology of the present invention, the term isocyanates encompasses all isocyanates known in the specialist field in connection with urethane chemistry, such as, in particular, phenyl isocyanate (PHI, obtainable by phosgenation of aniline, ANL), tolylene diisocyanate (TDI; obtainable by phosgenation of tolylenediamine, TDA), the di- and polyisocyanates of the diphenylmethane series (MDI; obtainable by phosgenation of the di- and polyamines of the diphenylmethane series, MDA), pentane 1,5-diisocyanate (PDI; obtainable by phosgenation of pentane-1,5-diamine, PDA), hexamethylene 1,6-diisocyanate (HDI; obtainable by phosgenation of hexamethylene-1,6-diamine, HDA), isophorone diisocyanate (IPDI; obtainable by phosgenation of isophoronediamine, IPDA) and xylylene diisocyanate (XDI; obtainable by phosgenation of xylylenediamine, XDA). The expression “an isocyanate” does of course also encompass embodiments in which two or more different isocyanates (e.g. mixtures of MDI and TDI) were used in the production of the (poly)urethane, unless explicitly stated otherwise, for instance by the wording “exactly one isocyanate”. The entirety of all isocyanates used in the production of the (poly)urethane is referred to as the isocyanate component (of the (poly)urethane). The isocyanate component comprises at least one isocyanate. Analogously, the entirety of all mono- or polyols used in the production of the (poly)urethane is referred to as the alcohol component (of the (poly)urethane). The alcohol component comprises at least one mono- or polyol.

In the terminology of the present invention, the term mono- or polyols encompasses all mono- or polyols known in the specialist field in connection with urethane chemistry, such as, in particular, polyether monools, polyether polyols, polyester polyols, polyetherester polyols and polyethercarbonate polyols. The expression “a monool” or “a polyol” do of course also encompass embodiments in which two or more different mono- or polyols were used in the production of the urethane. Therefore, if reference is made hereinafter, for example, to “a polyether polyol” (or “a polyester polyol” etc.), this terminology does of course also encompass embodiments in which two or more different polyether polyols (or two or more different polyester polyols etc.) were used in the production of the (poly)urethane.

In the terminology of the present invention, carbamates refer to the urethanes formed in the case of performance of the chemolysis as alcoholysis or hydroalcoholysis as a result of the reaction with the alcohol, in order to be able to distinguish them from the urethane used.

An amine corresponding to an isocyanate is the amine that can be phosgenated to obtain the isocyanate according to R—NH₂+COCl₂→R—N═C═O+2 HCl. Analogously, a nitro compound corresponding to an amine is the nitro compound that can be reduced to obtain the amine according to R—NO₂+3 H₂→R—NH₂+2 H₂O.

In the method of the invention, the water and alcohol chemolysis reagents are used in superstoichiometric amounts. This means that, in the case of hydrolysis, water is used in an amount theoretically sufficient to hydrolyze all the urethane bonds of the (poly)urethane to give amines and mono- or polyols with release of carbon dioxide. Similarly, the superstoichiometric use of alcohol means that said alcohol in the case of alcoholysis is used in an amount theoretically sufficient to convert all the urethane bonds of the (poly)urethane to form carbamates of the alcohol and mono- or polyols. In the case of hydroalcoholysis, alcohol and water are each used in an amount theoretically sufficient to hydrolyze all the urethane bonds of the (poly)urethane to amines and polyols, or to form carbamates of the alcohol and mono- or polyols, with release of carbon dioxide.

### Chemolysis Methods (Ia), (Ib) and (Ic)

In the context of the present invention, an alcoholysis (Ia) refers to a chemolysis using (at least) an alcohol without (deliberate) addition of water as chemolysis reagent. Since alcohols are frequently not entirely anhydrous (unless they are dried and stored with exclusion of moisture until use), it is possible for that reason for small amounts of water to be present in an alcoholysis for the purposes of the invention even though water is not deliberately used as chemolysis reagent. However, water, in an alcoholysis for the purposes of the invention, is introduced into the chemolysis at most in such an amount that the mass of water present during the alcoholysis is 0% to <4.0%, especially 0% to 3.5%, preferably 0% to 3.4%, more preferably 0% to 3.0%, most preferably 0% to 2.0%, of the mass of the alcohol used. The water content of alcohol is determinable by Karl Fischer titration; this is the crucial method for the purposes of the present invention. Karl Fischer titration has been described many times and is well known to the person skilled in the art. Various possible configurations of the basic principle of Karl Fischer titration generally give results with sufficiently good agreement within the scope for the purposes of the present invention. In the case of doubt, Karl Fischer titration as described in DIN 51 777, Part 1, March 1983, is crucial for the purposes of the present invention. In ascertaining the amount of alcohol to be used and the maximum permissible amount of water, the crucial figure is fundamentally the mass of the “pure” alcohol (=alcohol minus water present therein). However, if the alcohol contains only traces of water, it is possible, as will be immediately apparent to the person skilled in the art, to neglect this water content without making a significant error in ascertaining the amount of alcohol to be used and the maximum permissible amount of water. In the case of doubt, however, the crucial figure is the mass of the alcohol as such, i.e. minus the mass of water present therein, and the amount of water present in the alcohol should be taken into account in ascertaining the maximum permissible amount of water. If only an “excessively moist” alcohol is available and the chemolysis is nevertheless to be conducted as an alcoholysis (and not as a hydroalcoholysis), the alcohol may be freed of excess water by drying methods that are known per se.

In the context of the present invention, a hydrolysis (Ib) refers to a chemolysis using solely water as chemolysis reagent. By contrast with alcoholysis, the use of water without unintentional inclusion of further chemolysis reagents is directly possible.

In the context of the present invention, a hydroalcoholysis (Ic) refers to a chemolysis using alcohol and water (without further chemolysis reagents), where the mass of water accounts for at least 4.0%, preferably 4.0% to 15%, more preferably 4.0% to 10%, with very particular preference 5.0% to 10% and very exceptionally preferably 5.0% to 7.0% of the mass of the alcohol. With regard to the quantification of the total amount of water present when moist alcohols are used, the statements made above with regard to the alcoholysis are correspondingly applicable. In practice, it will therefore regularly be sufficient to take account solely of the mass of water added deliberately. If, as a result of a significant water content in the alcohol used, contrary to expectation, there are doubts as to whether the water content thus ascertained is within the aforementioned ranges, the water content of the alcohol should be determined as set out above for the alcoholysis (Ia) and taken into account. Alcohol and water may, but do not have to, be added simultaneously to the urethane to be cleaved. A hydroalcoholysis for the purposes of the invention includes addition firstly of the alcohol only to the urethane in order to bring it into solution as far as possible (in which case there will already be transurethanizations), and only then addition of water to the process product thus obtained.

With regard to the salt of an oxoacid of an element of the fifth, fourteenth or fifteenth group of the Periodic Table of the Elements (henceforth: salt of the oxoacid for short), it is unnecessary for the purposes of the present invention for the oxoacid as such to be a stable, isolable compound. For example, carbonates can be derived formally from “carbonic acid H₂CO₃”; the fact that this is not isolable in pure form is not a barrier to this and does not leave the scope of the present invention.

The ending “ate” in the context of the present invention serves to identify salts and does not denote esters. For example, the term “alkylphosphonate” denotes a salt having the anion RP(O)O₂²⁻, which can be derived from alkylphosphonic acid, RP(O)(OH)₂, by the complete deprotonation thereof. In the case of salts that can be derived from polyprotic acids by partial deprotonation thereof, the number of remaining hydrogens is stated explicitly, for example dihydrogenorthosilicate for H₂SiO₄²⁻. However, the precursor “mono” in the case of a single remaining hydrogen atom can be omitted, for example hydrogenorthosilicate (rather than monohydrogenorthosilicate) for HSiO₄³⁻.

Determination of the pK_{B} values

pK_{B} values in the context of the present invention are understood to mean the pK_{B} values in “ideally dilute” aqueous solution, i.e. the pK_{B} values in the case of negligible interaction between cation and anion of the salt of the oxoacid, in the temperature range from 23° C. to 25° C. The following equation known for corresponding acid-base pairs is applicable here with sufficient accuracy: pK_{A}+pK_{B}=14.00. Consequently, for example, the pK_{B} of all hydroxides (irrespective of the counterion), for the purposes of the present invention, is equated to 0.00 and is therefore not within the inventive range from 0.10 to 6.00. The pK_{A} values of numerous oxoacids and hence also the pK_{B} values of their salts (via pK_{A}+pK_{B}=14.00) are known from the literature. Reference is made here in particular to the standard textbook “Holleman, Arnold F.; Wiberg, Egon; Wiberg, Nils: Inorganic chemistry, 101st edition, De Gruyter”, where numerous pK_{A} values of oxoacids are reported in the chapters for the corresponding elements; for example: orthophosphoric acid, pK_{A} of the third dissociation stage=12.3 (p. 771); orthosilicic acid, pK_{A} of the second dissociation stage=11.7 (p. 923) and “carbonic acid”, pK_{A} of the second dissociation stage=10.3 (p. 862).

If it is not possible to make use of literature values, the pK_{B} is determined in the context of the present invention by acid-base titration. This is effected by analytical determination of the base constant (K_{B}) of the anion of the oxoacid and calculation of the pK_{B} therefrom. The procedure for such an acid-base titration is known to the person skilled in the art. Reference is made to the relevant technical literature such as, in particular, “Gerhart Jander, Karl Friedrich Jahr, Gerhard Schulze, Jürgen Simon (ed.): Maßanalyse. Theorie und Praxis der Titrationen mit chemischen und physikalischen Indikationen [Quantitative Analysis: Theory and Practice of Titrations with Chemical and Physical Indications], 16th edition, Walter de Gruyter, Berlin 2003, pages 67 to 128”. The base constants K_{B} are calculated with the aid of the equation for the hydroxide ion concentration given in the chapter “Sehr schwache Sauren und Basen” [Very Weak Acids and Bases] (pages 86 and 87). Solving this equation for K_{B} gives:

$${K}_{B} = [ {c}^{2} ( {OH}^{-} ) - {K}_{W} ] / {c}_{0} ( B ) \qquad ( I )$$

in which c(OH⁻) denotes the concentration of hydroxide ions determined by titration with an acid, K_{W} the ionic product of water (10⁻¹⁴ mol²/l²), and co(B) the starting concentration of the base (=of the anion of the oxoacid), i.e. the concentration calculated from the starting weight. In many cases, especially in the lower region of the inventive pK_{B} range from 0.10 to 6.00, the influence of autoprotolysis of water is very small, and so it is also possible to calculate using the simplified equation

$${K}_{B} = {c}^{2} ( {OH}^{-} ) / {c}_{0} ( B ) ; \qquad ( II )$$

in the case of doubt, however, for the purposes of the invention, the exact equation (I) is crucial. For the purposes of the present invention, the titration is conducted with 0.1 N hydrochloric acid using phenolphthalein.

There will initially follow a brief summary of various possible embodiments of the invention:

In a first embodiment of the method of the invention, which can be combined with all other embodiments, the method additionally comprises a step of
(C) workup of the chemolysis product to obtain a first product phase comprising mono- and/or polyols (namely the alcohol component and/or other alcohols formed therefrom in the chemolysis) and a second product phase comprising (i), in the case of performance of the chemolysis as alcoholysis (Ia), carbamates (optionally together with small amounts of amines that have formed, for example, as a result of the presence of traces of water in the alcohol), or (ii), in the case of performance of the chemolysis as hydrolysis (Ib) or hydroalcoholysis (Ic), amines.

In a second embodiment of the method of the invention, which is a particular configuration of the first embodiment, step (C) comprises a phase separation of the chemolysis product into the first product phase and the second product phase.

In a third embodiment of the method of the invention, which is a further particular configuration of the first embodiment, the method comprises the performance of the chemolysis as hydrolysis (Ib), wherein step (C) comprises blending the chemolysis product with an organic solvent and phase separation into the first product phase and the second product phase.

In a fourth embodiment of the method of the invention, which is a further particular configuration of the first embodiment, the method comprises the performance of the chemolysis as alcoholysis (Ia) or hydroalcoholysis (Ic), wherein step (C) comprises blending the chemolysis product with an organic solvent incompletely miscible with the alcohol used in step (B), and phase separation into the first product phase and the second product phase.

In a fifth embodiment of the method of the invention, which is a further particular configuration of the first embodiment, the method comprises the performance of the chemolysis as alcoholysis (Ia) or hydroalcoholysis (Ic), wherein step (C) comprises:
(C.I) mixing the chemolysis product with an organic solvent miscible with the alcohol used in step (B) to obtain a product mixture, and
(C.II) washing the product mixture with an aqueous washing liquid and phase separation into the first product phase and the second product phase.

In a sixth embodiment of the method of the invention, which is a particular configuration of the first embodiment and can be combined with all other configurations of that embodiment, the method comprises step (D), the obtaining of the mono- and/or polyols from the first product phase.

In a seventh embodiment of the method of the invention, which is a particular configuration of the sixth embodiment, step (D) comprises a distillation and/or stripping.

In an eighth embodiment of the method of the invention, which is a particular configuration of the first embodiment and can be combined with all other configurations of that embodiment, the method comprises step (E), the obtaining of the amines from the second product phase.

In a ninth embodiment of the method of the invention, which is a particular configuration of the eighth embodiment, the method comprises the performance of the chemolysis as alcoholysis (Ia), wherein step (E) comprises a hydrolysis of the carbamates to amines and a distillative removal of alcohol and water, followed by a distillative purification of the amines remaining after the distillative removal.

In a tenth embodiment of the method of the invention, which is a further particular configuration of the eighth embodiment, the method comprises the performance of the chemolysis as hydrolysis (Ib) or hydroalcoholysis (Ic), wherein step (E) comprises a distillative removal of alcohol and water from the second product phase, followed by a distillative purification of the amines remaining after the distillative removal.

In an eleventh embodiment of the method of the invention, which is combinable with all other embodiments (except for those that are limited to a hydrolysis (Ib)), the method comprises the performance of the chemolysis as alcoholysis (Ia) or hydroalcoholysis (Ic), wherein the alcohol used in the chemolysis is selected from methanol, ethanol, ethylene glycol, diethylene glycol, propylene glycol, dipropylene glycol, methylglycol, triethylene glycol, glycerol, 2-methylpropane-1,3-diol or a mixture of two or more of the aforementioned alcohols.

In a twelfth embodiment of the method of the invention, which is combinable with all other embodiments, the element from the fifth, fourteenth or fifteenth group of the Periodic Table of the Elements is selected from vanadium, carbon, silicon and phosphorus.

In a thirteenth embodiment of the method of the invention, which is a particular configuration of the twelfth embodiment, the method comprises the performance of the chemolysis as alcoholysis (Ia), wherein the salt of the oxoacid comprises an anion selected from
orthovanadate (VO₄³⁻),
orthophosphate (PO₄³⁻),
diphosphate (P₂O₇⁴⁻),
triphosphate (P₃O₉⁵⁻),
tetraphosphate (P₄O₁₁⁶⁻),
metaphosphate ([PO₃)⁻]ₙ),
alkylphosphonate (RP(O)O₂²⁻; in which R denotes an alkyl radical having 1 to 18 carbon
atoms, preferably 1 to 10 carbon atoms),
arylphosphonate (ArP(O)O₂²⁻; in which Ar denotes an aryl radical, especially phenyl),
hydrogenorthosilicate (HSiO₄³⁻),
metasilicate ([SiO₃²⁻]ₙ),
hydrogenmetasilicate ([HSiO₃⁻]ₙ),
dihydrogenorthosilicate (H₂SiO₄²⁻),
trihydrogenorthosilicate (H₃SiO₄—),
alkylsilanolate (RₓSiO₄₋ₓ^{(4-x)-}; in which R denotes an alkyl radical having 1 to 18 carbon atoms, preferably 1 to 10 carbon atoms, and x is 1, 2 or 3 and preferably 1 or 2, more preferably 1) or
arylsilanolate (ArₓSiO₄₋ₓ^{(4-x)-}; in which Ar denotes an aryl radical, especially phenyl, and x is 1, 2 or 3 and preferably 1 or 2, more preferably 1).

In particular, the salt of the oxoacid does not comprise any further anions aside from the above. More preferably, the anion of the salt of the oxoacid is selected from the group consisting of orthophosphate and orthovanadate.

In a fourteenth embodiment of the method of the invention, which is a further particular configuration of the twelfth embodiment, the method comprises the performance of the chemolysis as hydrolysis (Ib), wherein the salt of the oxoacid comprises an anion selected from
orthovanadate (VO₄³⁻),
carbonate (CO₃²⁻),
orthophosphate (PO₄³⁻),
diphosphate (P₂O₇⁴⁻)
triphosphate (P₃O₉⁵⁻)
tetraphosphate (P₄O₁₁⁶⁻)
metaphosphate ([PO₃)⁻]ₙ),
alkylphosphonate (RP(O)O₂²⁻; in which R denotes an alkyl radical having 1 to 18 carbon atoms, preferably 1 to 10 carbon atoms),
arylphosphonate (ArP(O)O₂²⁻; in which Ar denotes an aryl radical, especially phenyl),
hydrogenorthosilicate (HSiO₄³⁻),
metasilicate ([SiO₃²⁻],),
hydrogenmetasilicate ([HSiO₃⁻]ₙ),
dihydrogenorthosilicate (H₂SiO₄²⁻),
trihydrogenorthosilicate (H₃SiO₄—),
alkylsilanolate (RₓSiO₄₋ₓ^{(4-x)-}; in which R denotes an alkyl radical having 1 to 18 carbon atoms, preferably 1 to 10 carbon atoms, and x is 1, 2 or 3 and preferably 1 or 2, more preferably 1), or
arylsilanolate (ArₓSiO₄₋ₓ^{(4-x)-}; in which Ar denotes an aryl radical, especially phenyl, and x is 1, 2 or 3 and preferably 1 or 2, more preferably 1).

In particular, the salt of the oxoacid does not comprise any further anions aside from the above.

In a fifteenth embodiment of the method of the invention, which is a further particular configuration of the twelfth embodiment, the salt of the oxoacid (irrespective of the chemolysis method chosen) comprises an anion selected from
orthovanadate (VO₄³⁻),
diphosphate (P₂O₇⁴⁻,
triphosphate (P₃O₉⁵⁻,
tetraphosphate (P₄O₁₁⁶⁻,
alkylphosphonate (RPO₃²⁻; in which R denotes an alkyl radical having 1 to 18 carbon atoms, preferably 1 to 10 carbon atoms),
arylphosphonate (ArPO₃²⁻; in which Ar denotes an aryl radical, especially phenyl),
hydrogenorthosilicate (HSiO₄³⁻),
metasilicate ([SiO₃²⁻]),
hydrogenmetasilicate ([HSiO₃⁻]ₙ),
dihydrogenorthosilicate (H₂SiO₄²⁻),
trihydrogenorthosilicate (H₃SiO₄⁻),
alkylsilanolate (RₓSiO₄₋ₓ^{(4-x)-}; in which R denotes an alkyl radical having 1 to 18 carbon atoms, preferably 1 to 10 carbon atoms, and x is 1, 2 or 3 and preferably 1 or 2, more preferably 1), or
arylsilanolate (ArₓSiO₄₋ₓ^{(4-x)-}; in which Ar denotes an aryl radical and x is 1, 2 or 3 and preferably 1 or 2, more preferably 1).

The aforementioned anions are especially suitable in the case of performance of the chemolysis as hydroalcoholysis (Ic). In particular, the salt of the oxoacid does not comprise any further anions aside from the above.

In a sixteenth embodiment of the method of the invention, which can be combined with all other embodiments, the salt of the oxoacid is an alkali metal salt or a quaternary ammonium salt.

In a seventeenth embodiment of the method of the invention, which is a particular configuration of the sixteenth embodiment, the alkali metal salt is a sodium or potassium salt.

In an eighteenth embodiment of the method of the invention, which can be combined with all other embodiments, the chemolysis is conducted at a pressure in the range from 200 mbar_{(abs)} to 50 bar_{(abs)}, preferably 500 mbar_{(abs)} to 50 bar_{(abs)}, more preferably 900 mbar_{(abs)} to 1.8 bar_{(abs)}, especially at ambient pressure.

In a nineteenth embodiment of the method of the invention, which can be combined with all other embodiments, the chemolysis is performed at a temperature in the range from 50° C. to 195° C., preferably from 80° C. to 150° C., more preferably 100° C. to 130° C., most preferably 110° C. to 120° C.

In a twentieth embodiment of the method of the invention, which can be combined with all other embodiments, the isocyanate component comprises an isocyanate selected from
phenyl isocyanate (PHI; prepared from aniline, ANL), tolylene diisocyanate (TDI; prepared from tolylenediamine, TDA), the di- and polyisocyanates of the diphenylmethane series (MDI; prepared from the di- and polyamines of the diphenylmethane series, MDA), pentane 1,5-diisocyanate (PDI; prepared from pentane-1,5-diamine, PDA), hexamethylene 1,6-diisocyanate (HDI; prepared from hexamethylene-1,6-diamine, HDA), isophorone diisocyanate (IPDI; prepared from isophoronediamine, IPDA), xylylene diisocyanate (XDI; prepared from xylylenediamine, XDA) or a mixture of two or more of the aforementioned isocyanates.

In a twenty-first embodiment of the method of the invention, which is a particular configuration of the twentieth embodiment, the isocyanate component comprises tolylene diisocyanate or a mixture of tolylene diisocyanate and the di- and polyisocyanates of the diphenylmethane series (and especially does not comprise any further isocyanates other than the aforementioned isocyanates).

In a twenty-second embodiment of the method of the invention, which can be combined with all other embodiments, the alcohol component comprises a monool or polyol selected from
a polyether monool, a polyether polyol, a polyester polyol, a polyetherester polyol, a polyacrylate polyol, a polyethercarbonate polyol or a mixture of two or more of the aforementioned polyols. The alcohol component preferably contains a polyether polyol. More preferably, the alcohol component is a polyether polyol (i.e. does not contain any monools or polyols other than polyether polyols; but a mixture of two or more different polyether polyols is encompassed and does not depart from the scope of this embodiment).

In a twenty-third embodiment of the method of the invention, which can be combined with all other embodiments, the alcohol component comprises a styrene-acrylonitrile copolymer-filled polyether polyol.

In a twenty-fourth embodiment of the method of the invention, which can be combined with all other embodiments, the mass of the salt of the oxoacid is 0.10% to 20%, preferably 1.0% to 15%, more preferably 5.0% to 10%, of the mass of the (poly)urethane.

In a twenty-fifth embodiment of the method of the invention, which can be combined with all other embodiments, the mass ratio of chemolysis reagent (used in total) to the (poly)urethane

$$( \frac{m ( chemolysis reagent )}{m ( ( poly ) urethane )} )$$

is in the range from 0.05 to 90, preferably 1.0 to 80.

In a twenty-sixth embodiment of the method of the invention, which can be combined with all other embodiments, the chemolysis is conducted in the presence of (at least) a phase transfer catalyst.

In a twenty-seventh embodiment of the method of the invention, which is a particular configuration of the twenty-sixth embodiment, the phase transfer catalyst comprises a charged organic molecule.

In a twenty-eighth embodiment of the method of the invention, which is a particular configuration of the twenty-seventh embodiment, the phase transfer catalyst comprises a quaternary ammonium salt, a quaternary phosphonium salt or a mixture of the two.

The embodiments outlined briefly above and further possible configurations of the invention are more Particularly elucidated hereinbelow. All the above-described embodiments and the further configurations of the invention described below are mutually and collectively combinable as desired unless the opposite is clearly apparent from the context to a person skilled in the art or is expressly stated.

The method of the invention especially comprises a step of workup (II) of the method product from the chemolysis (I) to obtain (at least) a raw material selected from (a) a mono- and/or polyol, (b) a carbamate and/or (c) an amine. This workup (II) is preferably effected in a step (C) in which a first product phase comprising mono- and/or polyols (namely the alcohol component and/or other alcohols formed therefrom in the chemolysis) and a second product phase comprising (i), in the case of performance of the chemolysis as alcoholysis (Ia), carbamates (optionally together with small amounts of amines that have formed, for example, as a result of the presence of traces of water in the alcohol), or (ii), in the case of performance of the chemolysis as hydrolysis (Ib) or hydroalcoholysis (Ic), amines are obtained. In this preferred embodiment, the method of the invention therefore comprises the following steps:
(A) providing the urethane (especially polyurethane) based on an isocyanate component and an alcohol component;
(B) performing the chemolysis as described above, i.e. with the addition of a catalyst as one of the following reactions:
   (Ia) alcoholysis of the (poly)urethane with an alcohol (without deliberate addition of water);
   (Ib) hydrolysis of the (poly)urethane with water (without addition of an alcohol) or
   (Ic) hydroalcoholysis of the (poly)urethane with an alcohol and water,
   wherein the catalyst comprises a salt of an oxoacid of an element from the fifth, fourteenth or fifteenth group of the Periodic Table of the Elements or a mixture of two or more such salts, where the pK_{B} of the anion of the salt is in the range from 0.10 to 6.00, preferably 0.25 to 5.00, more preferably 0.50 to 4.50, and wherein the catalyst in the case of performance of the chemolysis as alcoholysis (Ia) does not comprise any carbonate and the catalyst in the case of performance of the chemolysis as hydroalcoholysis (Ic) does not comprise any carbonate, any orthophosphate or any metaphosphate,
   to obtain a chemolysis product;
and
(C) workup of the chemolysis product to obtain a first product phase comprising mono- and/or polyols (namely the alcohol component and/or other alcohols formed therefrom in the chemolysis) and a second product phase comprising (i), in the case of performance of the chemolysis as alcoholysis (Ia), carbamates (optionally together with small amounts of amines that have formed, for example, as a result of the presence of traces of water in the alcohol), or (ii), in the case of performance of the chemolysis as hydrolysis (Ib) or hydroalcoholysis (Ic), amines.

The carbamates are other urethanes formed by transurethanization reactions, while the amines are amines corresponding to the isocyanate that are formed by hydrolysis reactions, as defined further up.

### Provision of the (Poly)Urethane for Chemical Recycling

Step (A) of the aforementioned embodiment comprises providing the (poly)urethane to be chemically recycled in preparation for the chemolysis. This may in principle be any kind of urethane.

Preference is given to polyurethanes, i.e. urethanes that derive from polyisocyanates (2 or more isocyanate groups per molecule) and polyols (two or more alcohol groups per molecule). This may in principle be any kind of polyurethane, i.e. either polyurethane foams or polyurethane products from the so-called CASE applications described at the outset. The polyurethane foams may be either flexible foams or rigid foams, preference being given to flexible foams (for example from used mattresses, furniture cushioning or car seats). Polyurethane foams are typically produced using blowing gases such as pentane or carbon dioxide. In the case of polyurethanes from CASE applications, preference is given to polyurethane elastomers, polyurethane adhesives and polyurethane coatings.

With regard to the isocyanate component, preference is given to those urethanes or polyurethanes comprising an isocyanate selected from
phenyl isocyanate (PHI; prepared from aniline, ANL), tolylene diisocyanate (TDI; prepared from tolylenediamine, TDA), the di- and polyisocyanates of the diphenylmethane series (MDI; prepared from the di- and polyamines of the diphenylmethane series, MDA), pentane 1,5-diisocyanate (PDI; prepared from pentane-1,5-diamine, PDA), hexamethylene 1,6-diisocyanate (HDI; prepared from hexamethylene-1,6-diamine, HDA), isophorone diisocyanate (IPDI; prepared from isophoronediamine, IPDA), xylylene diisocyanate (XDI; prepared from xylylenediamine, XDA) or a mixture of two or more of the aforementioned isocyanates.

In the case of the polyurethanes, it is particularly preferable that the isocyanate component comprises tolylene diisocyanate or a mixture of tolylene diisocyanate and the di- and polyisocyanates of the diphenylmethane series, and especially does not comprise any further isocyanates aside from the above.

With regard to the alcohol component, this preferably comprises a mono- or polyol selected from
a polyether monool, a polyether polyol, a polyester polyol, a polyetherester polyol, a polyacrylate polyol, a polyethercarbonate polyol or a mixture of two or more of the aforementioned polyols.

The alcohol component preferably contains a polyether polyol. More preferably, the alcohol component is a polyether polyol (i.e. does not contain any mono- or polyols other than polyether polyols; but a mixture of two or more different polyether polyols is encompassed and does not depart from the scope of this embodiment).

The polyether polyol may also be one that is filled with a styrene-acrylonitrile copolymer (SAN copolymer). In such cases, it is advantageous to conduct the chemolysis as hydrolysis (Ib) or hydroalcoholysis (Ic). The challenge in the chemolysis of polyurethanes having a polyol component based on SAN copolymer-filled polyether polyols is that the SAN copolymer is released as finely divided polymer particles during the chemolysis. The SAN polymer present as finely divided polymeric particles in the reaction mixture leads to problems in the subsequent separation by extractive methods for example. Furthermore, due to the fineness of the polymer particles, filtration is hardly possible since the filter quickly becomes blocked and further removal is no longer possible. When the chemolysis is performed as a hydrolysis (Ib) or hydroalcoholysis (Ic), the SAN polymer, after it has been released from the polyether polyol, is partly converted back to a soluble form by the hydrolysis step, which facilitates the workup of the reaction mixture by an extraction after the chemolysis.

Preferably, even step (A) comprises preparatory steps for the cleavage of the urethane bonds in step (B). In the case of polyurethanes, these are especially mechanical comminution. Such preparatory steps are known to a person skilled in the art; reference is made by way of example to the literature cited in [1]. Depending on the characteristics of the polyurethane (especially in the case of polyurethane foams), it can be advantageous to “freeze” this before the mechanical comminution in order to facilitate the comminuting operation.

Before, during or after the mechanical comminution, the polyurethane can be treated with aqueous or alcoholic disinfectants. Such disinfectants are preferably hydrogen peroxide, chlorine dioxide, sodium hypochlorite, formaldehyde, sodium N-chloro-(4-methylbenzene)sulfonamide (chloramine T) and/or peracetic acid (aqueous disinfectants) or ethanol, isopropanol and/or 1-propanol (alcoholic disinfectants).

It is also conceivable to conduct the above-described preparatory steps at a site spatially separate from the site of the chemolysis. In that case, the prepared foam is transferred into suitable transport vehicles, for example silo vehicles, for further transport. For further transport the prepared foam may additionally be compressed to achieve a higher mass-to-volume ratio.

The foam is then transferred into the reaction apparatus provided for the chemolysis at the location of the chemolysis. It is also conceivable to connect the transport vehicle used directly to the reaction apparatus.

### Chemolysis of the (Poly)Urethane

The chemolysis of the (poly)urethane, step (B), is preferably conducted with exclusion of oxygen. This means that the reaction is carried out in an inert gas atmosphere (especially in a nitrogen, argon or helium atmosphere). Preference is also given to freeing the chemolysis reagents used (alcohol, water, or alcohol and water) of oxygen by inert gas saturation.

According to the invention, the chemolysis can be conducted as alcoholysis (Ia), hydrolysis (Ib) or hydroalcoholysis (Ic). The terms “alcoholysis” and “hydroalcoholysis” used here are usually referred to in the literature respectively as glycolysis and hydroglycolysis; cf. No. 2 and 3 further up. However, since this is only truly correct when glycol is used as alcohol, the more general terms alcoholysis and hydroalcoholysis are used in the context of the present invention. In the case of performance of the chemolysis as alcoholysis (Ia) or hydroalcoholysis (Ic), in the method of the invention, the alcohol used in the chemolysis is preferably selected from methanol, ethanol, ethylene glycol, diethylene glycol, propylene glycol, dipropylene glycol, methylglycol, triethylene glycol, glycerol, 2-methylpropane-1,3-diol or a mixture of two or more of the aforementioned alcohols. Diethylene glycol and propylene glycol are particularly preferred. In the case of hydroglycolysis, it is possible to premix water and alcohol; but this is not obligatory.

According to the invention, the catalyst used for the chemolysis is a salt of an oxoacid of an element from the fifth, fourteenth or fifteenth group of the Periodic Table of the Elements or a mixture of two or more such acids, where the pK_{B} of the anion of the salt is in the range from 0.10 to 6.00, preferably 0.25 to 5.00, more preferably 0.50 to 4.50, and wherein the catalyst in the case of performance of the chemolysis as alcoholysis (Ia) does not comprise any carbonate and the catalyst in the case of performance of the chemolysis as hydroalcoholysis (Ic) does not comprise any carbonate, any orthophosphate or any metaphosphate. It is preferable here that the element chosen from the fifth, fourteenth or fifteenth group of the Periodic Table is vanadium, carbon, silicon or phosphorus. In the case of performance of the chemolysis as alcoholysis (Ia), the salt of the oxoacid preferably comprises an anion selected from
orthovanadate (VO₄³⁻),
orthophosphate (PO₄³⁻),
diphosphate (P₂O₇⁴⁻),
triphosphate (P₃O₉⁵⁻),
tetraphosphate (P₄O₁₁⁶⁻),
metaphosphate ([PO₃)⁻]ₙ),
alkylphosphonate (RP(O)O₂²⁻; in which R denotes an alkyl radical having 1 to 18 carbon atoms, preferably 1 to 10 carbon atoms),
arylphosphonate (ArP(O)O₂²⁻; in which Ar denotes an aryl radical, especially phenyl),
hydrogenorthosilicate (HSiO₄³⁻),
metasilicate ([SiO₃²⁻]),
hydrogenmetasilicate ([HSiO₃⁻]ₙ),
dihydrogenorthosilicate (H₂SiO₄²⁻),
trihydrogenorthosilicate (H₃SiO₄⁻),
alkylsilanolate (RₓSiO₄₋ₓ^{(4-x)-}; in which R denotes an alkyl radical having 1 to 18 carbon atoms, preferably 1 to 10 carbon atoms, and x is 1, 2 or 3 and preferably 1 or 2, more preferably 1), or
arylsilanolate (ArₓSiO₄₋ₓ^{(4-x)-}; in which Ar denotes an aryl radical, especially phenyl, and x is 1, 2 or 3 and preferably 1 or 2, more preferably 1).

Of the aforementioned anions, particular preference is given to orthovanadate (VO₄³⁻), orthophosphate (PO₄³⁻) and hydrogenorthosilicate (HSiO₄³⁻). Very particular preference is given to orthovanadate (VO₄³⁻) and orthophosphate (PO₄³⁻).

In the case of performance of the chemolysis as hydrolysis (Ib), the salt of the oxoacid preferably comprises an anion selected from
orthovanadate (VO₄³⁻),
carbonate (CO₃²⁻),
orthophosphate (PO₄³⁻),
diphosphate (P₂O₇⁴⁻),
triphosphate (P₃O₉⁵⁻),
tetraphosphate (P₄O₁₁⁶⁻),
metaphosphate ([PO₃)⁻]ₙ),
alkylphosphonate (RP(O)O₂²⁻; in which R denotes an alkyl radical having 1 to 18 carbon atoms, preferably 1 to 10 carbon atoms),
arylphosphonate (ArP(O)O₂²⁻; in which Ar denotes an aryl radical, especially phenyl),
hydrogenorthosilicate (HSiO₄³⁻)
metasilicate ([SiO₃²⁻]),
hydrogenmetasilicate ([HSiO₃⁻]ₙ),
dihydrogenorthosilicate (H₂SiO₄²⁻),
trihydrogenorthosilicate (H₃SiO₄⁻),
alkylsilanolate (RₓSiO₄₋ₓ^{(4-x)-}; in which R denotes an alkyl radical having 1 to 18 carbon atoms, preferably 1 to 10 carbon atoms, and x is 1, 2 or 3 and preferably 1 or 2, more preferably 1), or
arylsilanolate (ArₓSiO₄₋ₓ^{(4-x)-}; in which Ar denotes an aryl radical, especially phenyl, and x is 1, 2 or 3 and preferably 1 or 2, more preferably 1).

Of the aforementioned anions, particular preference is given to orthovanadate (VO₄³⁻), carbonate (CO₃²⁻), orthophosphate (PO₄³⁻) and hydrogenorthosilicate (HSiO₄³⁻). Very particular preference is given to orthovanadate (VO₄³⁻), carbonate (CO₃²⁻) and orthophosphate (PO₄³⁻), especially in conjunction with a phase transfer catalyst (see below for details).

In the case of all the described chemolysis methods (Ia) to (Ic), especially in the case of performance of the chemolysis as hydroalcoholysis (Ic), the salt of the oxoacid may be an anion selected from
orthovanadate (VO₄³⁻),
diphosphate (P₂O₇⁴⁻),
triphosphate (P₃O₉⁵⁻),
tetraphosphate (P₄O₁₁⁶⁻),
alkylphosphonate (RPO₃²⁻; in which R denotes an alkyl radical having 1 to 18 carbon atoms, preferably 1 to 10 carbon atoms),
arylphosphonate (ArPO₃²⁻; in which Ar denotes an aryl radical, especially phenyl),
hydrogenorthosilicate (HSiO₄³⁻),
metasilicate ([SiO₃²⁻]),
hydrogenmetasilicate ([HSiO₃⁻]ₙ),
dihydrogenorthosilicate (H₂SiO₄²⁻),
trihydrogenorthosilicate (H₃SiO₄⁻),
alkylsilanolate (RₓSiO₄₋ₓ^{(4-x)-}; in which R denotes an alkyl radical having 1 to 18 carbon atoms, preferably 1 to 10 carbon atoms, and x is 1, 2 or 3 and preferably 1 or 2, more preferably 1), or
arylsilanolate (ArₓSiO₄₋ₓ^{(4-x)-}; in which Ar denotes an aryl radical and x is 1, 2 or 3 and preferably 1 or 2, more preferably 1).

Of the aforementioned anions, particular preference is given to orthovanadate (VO₄³⁻) and hydrogenorthosilicate (HSiO₄³⁻).

The following is applicable irrespective of the chemolysis method chosen: It is preferable to use only one (1) salt of an oxoacid as catalyst and not a mixture. It is further preferable that no further catalysts that have not been enumerated above are used either. It has been found to be useful to meter in the salt of the oxoacid such that its mass is 0.10% to 20%, preferably 1.0% to 15%, more preferably 5.0% to 10%, of the mass of the (poly)urethane to be converted. Finally, it is preferable to use an alkali metal salt as the salt of the oxoacid, especially a sodium or potassium salt, or a quaternary ammonium salt. Sodium salts or potassium salts are particularly preferred.

In the chemolysis, preference is given to observing a reaction temperature in the range from 50° C. to 195° C., more preferably from 80° C. to 150° C., even more preferably 100° C. to 130° C. and very exceptionally preferably 110° C. to 120° C. The chemolysis can be performed in an autoclave without pressure compensation, where pressures of up to 50 bar_{(abs)} can be established. But the reaction does not make any particular demands on pressure; it can likewise be conducted at ambient pressure or slightly reduced pressure (especially with a lower pressure limit of 200 mbar_{(abs)}, preferably 900 mbar_{(abs)}), which facilitates the removal of carbon dioxide formed. An only slightly elevated pressure up to 1.8 bar_{(abs)} in particular is likewise possible.

The chemolysis is generally ended within a period of 1.0 h to 48 h, preferably 1.5 h to 24 h, more preferably 2.0 h to 10 h, even more preferably 2.5 h to 6.0 h and very exceptionally preferably 3.0 h to 5.5 h; in other words, after a reaction time within this period, only slight further conversion at most, if any, takes place.

In a preferred embodiment, a mass ratio of chemolysis reagent (used overall) to the (poly)urethane,

$$\frac{m ( chemolysis reagent )}{m ( ( poly ) urethane )} ,$$

in the range from 0.05 to 90, preferably from 1.0 to 80, is used.

In the case of hydroalcoholysis (Ic), this amount should be divided between water and alcohol, where the mass of water accounts for at least 4.0%, preferably 4.0% to 15%, more preferably 4.0% to 10%, with very particular preference 5.0% to 10% and very exceptionally preferably 5.0% to 7.0% of the mass of the alcohol. It is advantageous here not to add the water to be used for the hydroalcoholysis right at the start of the reaction period. It has been found to be useful first to add solely the alcohol and to dissolve the (poly)urethane therein (in which case, of course, transurethanization reactions will already take place) before the water is added. It is also possible to add a small amount of the water, especially 2% to 4% of the total amount of water to be used in the chemolysis, to the (poly)urethane together with the alcohol. The addition of the water in the former case or of the majority thereof in the latter case is then preferably not effected all at once, but with a time delay over the further course of chemolysis over the reaction time. In the case of addition of the water with a time delay, the requirement that the “mass of water accounts for at least 4.0%, preferably 4.0% to 15%, more preferably 4.0% to 10%, with very particular preference 5.0% to 10% and very exceptionally preferably 5.0% to 7.0% of the mass of the alcohol” relates to the total amount of water added as part of the chemolysis reagent.

In the case of performance of the chemolysis as alcoholysis (Ia), no water is used as chemolysis reagent. The introduction of small amounts of water from other sources, especially coming from moisture in the alcohol, is not ruled out thereby. Likewise not excluded is dissolution of the catalyst in water. Water, in the context of an alcoholysis of the invention, is introduced into the chemolysis at most in such an amount that the mass of water present during the alcoholysis (no matter where it comes from) is 0% to <4.0%, especially 0% to 3.5%, preferably 0% to 3.4%, more preferably 0% to 3.0%, most preferably 0% to 2.0%, of the mass of the alcohol used. The alcoholysis at first gives carbamates as products alongside mono- and/or polyols. If the aim is the isolation of amines, these have to be subjected to a hydrolysis in a method step separate from the alcoholysis, which is elucidated in greater detail further down.

It may be advantageous, in the chemolysis, in addition to the catalyst (i.e. in addition to the chemolysis catalyst), to add at least one phase transfer catalyst in order to increase the yield and/or to shorten the reaction time. Without wishing to be tied to any theory, it is assumed that a phase transfer catalyst promotes the transport of the actual catalyst (the chemolysis catalyst) into the (hydrophobic) (poly)urethane and hence accelerates the degradation reaction. Suitable phase transfer catalysts are preferably compounds having a charged organic molecule, preferably quaternary ammonium salts ([R₄N]⁺X⁻) or quaternary phosphonium salts ([R₄P]⁺X⁻) having organic radicals (R) and a counterion (X⁻). Particular preference is given to quaternary ammonium salts having organic radicals and a counterion. The organic radicals (R) are preferably methyl, propyl, butyl, pentyl, hexyl, octyl, hexadecyl or stearyl or benzyl radicals, where the four radicals of a quaternary ammonium or phosphonium salt may each be different or the same. The counterion (X⁻) is preferably chloride, bromide, sulfate, chlorate or triflate. Suitable phase transfer catalysts are, for example, trimethylbenzylammonium chloride, tetra(1-propyl)ammonium chloride, tetra(1-butyl)ammonium chloride, tetra(1-pentyl)ammonium chloride, tetra(1-hexyl)ammonium chloride, dimethyldistearylammonium chloride, tetraphenylphosphonium chloride, hexa(1-decyl)tributylphosphonium chloride, methyltri(1-octyl)phosphonium chloride and/or methytri(1-octyl)ammonium chloride (“Aliquat 336”), preference being given to tetra(1-butyl)ammonium chloride, tetra(1-pentyl)ammonium chloride, tetra(1-hexyl)ammonium chloride and methyltri(1-octyl)ammonium chloride (“Aliquat 336”). The phase transfer catalyst used is most preferably tetra(1-hexyl)ammonium chloride and/or methyltri(1-octyl)ammonium chloride (“Aliquat 336”).

The chemolysis can be conducted in any reactor known for such a purpose in the specialist field. In particular—as well as the autoclaves already mentioned—suitable chemolysis reactors are stirred tanks (stirred reactors) and tubular reactors.

### Workup of the Chemolysis Product

The chemolysis (I) of the (poly)urethane affords a product mixture, the chemolysis product. In order to isolate the sought-after raw materials, this chemolysis product has to be worked up (II); this is preferably accomplished according to step (C) of the preferred embodiment specified above. The workup (II) according to step (C) has the aim of providing two product phases, one of which (henceforth: first product phase) contains mono- and/or polyols (namely the alcohol component and/or other alcohols formed therefrom in the chemolysis) and/or the second of which (henceforth: second product phase), depending on the nature of the chemolysis, contains the following:
(i) in the case of performance of the chemolysis as alcoholysis (Ia): carbamates (possibly together with small amounts of amines that have formed, for example, as a result of the presence of traces of water in the alcohol), or
(ii) in the case of performance of the chemolysis as hydrolysis (Ib) or hydroalcoholysis (Ic): amines.

It will be self-evident to the person skilled in the art that the separation into the two product phases need not necessarily proceed perfectly in the sense that all mono- or polyol goes into the first product phase and all carbamate or amine into the second product phase. If, for example, because of the prevailing solubility equilibria, small amounts of the amine get into the first product phase (or small amounts of the mono- or polyol into the second product phase), this of course does not leave the scope of this embodiment.

Depending in particular on the nature of the chemolysis reagent used, it is possible that the chemolysis product is obtained directly in biphasic form. This is regularly the case when the chemolysis is performed as hydrolysis (Ib) or hydroalcoholysis (Ic). Even when the chemolysis is performed as alcoholysis, this often occurs, depending on the choice of alcohol. If one of the two phases then contains the majority of the mono- or polyols and the other of the two phases the majority of the carbamates or amines, the first and second product phases can be obtained by a simple phase separation. The first product phase can then be sent directly to a further workup for isolation of the mono- or polyols (henceforth step (D)). Correspondingly, the second product phase can be sent directly to a further workup for isolation of the amines (henceforth step (E)). This embodiment is conceivable, for example, when TDI-based polyurethane foams are used and the chemolysis is performed as hydroalcoholysis with diethylene glycol as alcohol. The TDA formed, because of its water solubility, together with the likewise water-soluble diethylene glycol and unconverted water, forms the second product phase (an alcoholic-aqueous phase), while the recovered polyols form the first product phase (an organic phase). Whether this embodiment is employable can be readily determined via considerations by the person skilled in the art or simple preliminary tests.

However, it is also possible that a simple phase separation of the chemolysis product does not lead to a first product phase having a sufficient mono- or polyol content and a second product phase having a sufficient amine or carbamate content. In such a case, it is preferable to extract the chemolysis product in its entirety with an organic solvent. This is also the method of choice when the chemolysis product is in monophasic form. There are several options for such an extraction:

In a preferred embodiment, which is employable in the case of performance of the chemolysis as hydrolysis (Ib), the chemolysis product is extracted with an organic solvent and then separated into the first and second product phases. Suitable organic solvents are aliphatic hydrocarbons, alicyclic hydrocarbons, aromatic hydrocarbons, halogen-substituted aliphatic hydrocarbons, halogen-substituted alicyclic hydrocarbons, halogen-substituted aromatic hydrocarbons and mixtures of two or more of the aforementioned organic solvents.

In a preferred embodiment, which is employable in the case of performance of the chemolysis as alcoholysis (Ia) or hydroalcoholysis (Ic), the workup of the chemolysis product comprises the blending thereof with an organic solvent not completely miscible with the alcohol used in the chemolysis, and phase separation into the first product phase and the second product phase. The requirement that the organic solvent to be used in this extraction is not completely miscible with the alcohol used in the chemolysis means that—under the conditions of temperature and ratio of organic solvent to alcohol from the chemolysis that exist for the extraction—there must be a miscibility gap such that a phase separation becomes possible. This is the case, for example, when the organic solvent is selected from aliphatic hydrocarbons, alicyclic hydrocarbons, aromatic hydrocarbons and mixtures of two or more of the aforementioned organic solvents, and the alcohol to be used in the chemolysis is selected from methanol, ethanol, ethylene glycol, diethylene glycol, propylene glycol, dipropylene glycol, methylglycol, triethylene glycol, glycerol, 2-methylpropane-1,3-diol and mixtures of two or more of the aforementioned alcohols. In the case of doubt, it is easy to find out by simple preliminary tests whether or not there is a suitable miscibility gap.

In a further preferred embodiment, which is likewise employable in the case of performance of the chemolysis as alcoholysis (Ia) or hydroalcoholysis (Ic), the workup of the chemolysis product is likewise conducted by extraction, but using an organic solvent which is miscible with the alcohol used in the chemolysis. In this case, the workup comprises the steps of:
(1) mixing the chemolysis product with an organic solvent miscible with the alcohol used in the chemolysis to obtain a product mixture, and
(2) washing the product mixture with an aqueous washing liquid and phase separation into the first product phase and the second product phase.

The requirement that the organic solvent to be used in step (1) is miscible with the alcohol used in step (B) means that—under the conditions of temperature and ratio of organic solvent to alcohol from the chemolysis that exist for step (1)—a mixture of the organic solvent and the alcohol from the chemolysis does not spontaneously separate into two phases. This is the case, for example, when the organic solvent in step (1) is selected from halogen-substituted aliphatic hydrocarbons, halogen-substituted alicyclic hydrocarbons, halogen-substituted aromatic hydrocarbons and mixtures of two or more of the aforementioned organic solvents, and the alcohol to be used in the chemolysis is selected from methanol, ethanol, ethylene glycol, diethylene glycol, propylene glycol, dipropylene glycol, methylglycol, triethylene glycol, glycerol, 2-methylpropane-1,3-diol and mixtures of two or more of the aforementioned alcohols.

### Workup of the First Product Phase

The first product phase contains the mono- and/or polyols and is preferably worked up in very substantially pure form for isolation thereof (step (D) in the preferred embodiment specified further up). Such a workup is preferably performed by distillation and/or stripping with a stripping gas (such as, in particular, nitrogen or steam, preferably nitrogen). This involves performing a distillation, preferably in an evaporator selected from falling-film evaporators, thin-film evaporators, flash evaporators, rising-film evaporators, natural circulation evaporators, forced circulation evaporators or tank evaporators. It is particularly preferable for the distillation to be followed by a stripping operation with steam. Stripping with steam may be performed by passing steam through stripping columns known per se. However, stripping with steam can also be effected in such a way that water in liquid form is added to the first product phase (which has optionally already been prepurified in a distillation), followed by superheating (against a pressure adjusted by a pressure valve which is sufficient to keep the water in liquid form) and decompressing downstream of the pressure valve, as a result of which the water present in the polyol evaporates and has a stripping effect.

### Workup of the Second Product Phase

The second product phase contains the amines or carbamates and is preferably worked up in very substantially pure form for isolation of the amines (step (E) in the preferred embodiment specified further up).

If the second product phase does not contain carbamates at all, or does so only in negligible proportions (as is expected in the case of performance of the chemolysis as hydrolysis (Ib) or hydroalcoholysis (Ic)), the recovery of the amine advantageously at first comprises a distillative separation of alcohol and water from the second product phase. This can be accomplished by known distillation techniques. The remaining crude amine is preferably worked up further, especially by distillation.

If the second product phase contains predominantly carbamates (as is expected in the case of performance of the chemolysis as alcoholysis (Ia)), the recovery of the amine advantageously comprises a hydrolysis of the carbamates to the amines and a distillative removal of alcohol and water, followed by a distillative purification of the crude amines remaining after the distillative removal. The hydrolysis and evaporation of water and alcohol need not necessarily be effected in that sequence. It is also entirely possible first to evaporate an alcohol fraction (generally together with a portion of the water), then to hydrolyze it and finally to remove the remaining water in the step of distilling the crude amine. Suitable catalysts for a hydrolysis step in the workup of the second product phase are the catalysts suitable for hydrolyses from the prior art, and likewise the catalysts to be used in accordance with the invention in the chemolysis in step (B). The possibility of phase transfer catalysis mentioned further up in connection with step (B) can be used in the same way in a hydrolysis as well in the workup of the second product phase.

It is in any case preferable to integrate the recovery of the amine into the workup of newly produced amine by mixing the crude amine with a crude product fraction of the amine deriving from new manufacture of the same amine. This embodiment provides an economic and environmentally friendly outlet for impurities deriving from the polyurethane product. It is described in more detail in international patent application WO 2020/260387 A1.

The invention is more particularly elucidated hereinafter with reference to examples.

### EXAMPLES

The test reaction chosen was the cleavage of 2-(2-ethoxyethoxy)ethyl N-phenylcarbamate to (i) aniline or the carbamate formed from aniline and monoethylene glycol (MEG) and (ii) 2-(2-ethoxyethoxy)ethan-1-ol. In each case, 0.25 mmol of 2-(2-ethoxyethoxy)ethyl phenylcarbamate was converted in an autoclave. The catalysts and phase transfer catalysts used were commercially available products and were used without further purification.

The tables that follow summarize the experimental conditions and results:

TABLE 1
Overview of the test results for chemolysis in general

| | Catalyst | | | | | |
|---|---|---|---|---|---|---|
| | | Conc. | Chemolysis | T | Reaction time | Conversion^{[b]} |
| Ex.^{[a]} | Type | [mol %] | reagent | [° C.] | [h] | [%] |
| 1 (I) | sodium | 20 | H₂O (5.0 ml) | 110 | 24 | 20 |
| | metasilicate | | | | | |
| | hydrate | | | | | |
| 2 (I) | sodium | 10 | H₂O (5.0 ml) | 110 | 24 | 18 |
| | metasilicate | | | | | |
| | hydrate | | | | | |
| 3 (C) | Bu₄NCl | 10 | H₂O (5.0 ml) | 110 | 24 | 1 |
| 4 (I) | Na₃VO₄ | 20 | H₂O (5.0 ml) | 100 | 48 | 89 |
| 5 (I) | Na₃VO₄ | 5 | H₂O (5.0 ml) | 110 | 48 | 92 |
| 6 (I) | Na₃VO₄ | 10 | H₂O (5.0 ml) | 110 | 24 | 60 |
| 7 (I) | Na₂CO₃ | 10 | H₂O (5.0 ml) | 110 | 24 | 93 |
| 8 (I) | Na₃VO₄ | 10 | H₂O (5.0 ml) | 110 | 18 | 15 |
| 9 (I) | Na₃VO₄ | 20 | H₂O (5.0 ml) | 110 | 48 | 89 |
| 10 (I) | Na₃VO₄ | 20 | H₂O (5.0 ml) | 120 | 24 | 84 |
| 11 (I) | Na₂CO₃ | 10 | H₂O (1.0 ml) | 150 | 24 | 100 |
| 12 (I) | K₃PO₄ | 10 | MEG (1.0 ml) | 110 | 24 | 78 |
| 13 (I) | Na₃PO₄ | 10 | MEG (0.5 ml) | 110 | 24 | 20 |
| 14 (C) | — | — | MEG (0.5 ml) | 110 | 48 | Traces |
| 15 (I) | Na₃VO₄ | 20 | MEG (0.5 ml) | 110 | 48 | 81 |
| 16 (I) | Na₃VO₄ | 20 | MEG (0.5 ml) | 110 | 24 | 64 |
| 17 (I) | Na₂CO₃ | 20 | MEG (0.5 ml) | 110 | 24 | 95 |
| 18 (I) | K₃PO₄ | 20 | MEG (0.5 ml) | 110 | 24 | 96 |
| 19 (C) | K₂HPO₄ | 20 | MEG (0.5 ml) | 110 | 24 | 18 |
| 20 (I) | Na₃PO₄ | 20 | MEG (0.5 ml) | 110 | 24 | 96 |
| 21 (C) | Na₂HPO₄ | 20 | MEG (0.5 ml) | 110 | 24 | 7 |
| 22 (C) | — | — | MEG (0.5 ml) | 150 | 24 | Traces |
| 23 (I) | Na₃VO₄ | 10 | MEG (0.5 ml) | 150 | 24 | 100 |

^{[a]}(I) = inventive experiment; (C) = comparative experiment
^{[b]}conversion of 2-(2-ethoxyethoxy)ethyl N-phenylcarbamate, determined by gas chromatography

TABLE 2
Overview of the experimental results relating to
the influence of phase transfer catalysts (PTC)

| | | | PTC | | | | Reaction | |
|---|---|---|---|---|---|---|---|---|
| | Type | Conc. | | Conc. | Chemolysis | T | time | Conversion^{[a]} |
| Ex. | Catalyst | [mol %] | Type | [mol %] | reagent | [° C.] | [h] | [%] |
| 24 | K₃PO₄ | 20 | — | — | H₂O (1.0 ml) | 110 | 17 | 21 |
| 25 | K₃PO₄ | 20 | — | — | H₂O (1.0 ml) | 110 | 24 | 29 |
| 26 | K₃PO₄ | 20 | Aliquat 336^{[b]} | 20 | H₂O (1.0 ml) | 110 | 17 | 73 |
| 27 | K₃PO₄ | 20 | (1-propyl)₄NCl | 20 | H₂O (1.0 ml) | 110 | 17 | 23 |
| 28 | K₃PO₄ | 20 | (1-butyl)₄NCl | 20 | H₂O (1.0 ml) | 110 | 17 | 30 |
| 29 | K₃PO₄ | 20 | (1-pentyl)₄NCl | 20 | H₂O (1.0 ml) | 110 | 17 | 55 |
| 30 | K₃PO₄ | 20 | (1-hexyl)₄NCl | 20 | H₂O (1.0 ml) | 110 | 17 | 81 |
| 31 | K₃PO₄ | 20 | — | — | MEG (1.0 ml) | 110 | 17 | 75 |
| 32 | K₃PO₄ | 20 | Aliquat 336^{[b]} | | MEG (1.0 ml) | 110 | 17 | 85 |

^{[a]}conversion of 2-(2-ethoxyethoxy)ethyl phenylcarbamate, determined by gas chromatography
^{[b]}methyltri(1-octyl)ammonium chloride

1. A method of cleaving urethanes by chemolysis (I), comprising (A) providing a urethane based on an isocyanate component and an alcohol component, followed by (B) chemolysis (I) of the urethane with a chemolysis reagent to obtain a chemolysis product, wherein the chemolysis (I) is conducted with addition of a catalyst as one of the following conversions:
(Ia) alcoholysis of the urethane, where the chemolysis reagent is an alcohol,
(Ib) hydrolysis of the urethane, where the chemolysis reagent is water,
or
(Ic) hydroalcoholysis of the urethane, where the chemolysis reagent comprises alcohol and water,
wherein the catalyst comprises a salt of an oxoacid of an element from the fifth, fourteenth or fifteenth group of the Periodic Table of the Elements or a mixture of two or more such acids, where the pK_{B} of the anion of the salt of the oxoacid is in the range from 0.10 to 6.00, and wherein the catalyst in the case of performance of the chemolysis as alcoholysis (Ia) does not comprise any carbonate and the catalyst in the case of performance of the chemolysis as hydroalcoholysis (Ic) does not comprise any carbonate, any orthophosphate or any metaphosphate.

2. The method as claimed in claim 1, further comprising:
(C) working up (II) the chemolysis product to obtain a first product phase comprising mono- and/or polyols and a second product phase comprising:
(i), in the case of performance of the chemolysis as alcoholysis (Ia), carbamates, or
(ii), in the case of performance of the chemolysis as hydrolysis (Ib) or hydroalcoholysis (Ic), amines.

3. The method as claimed in claim 2, in which step (C) comprises a phase separation of the chemolysis product into the first product phase and the second product phase.

4. The method as claimed in claim 2, in which
(i) in the case of performance of the chemolysis as hydrolysis (Ib), step (C) comprises blending the chemolysis product with an organic solvent and phase separation into the first product phase and the second product phase,
or
(ii) in the case of performance of the chemolysis as alcoholysis (Ia) or hydroalcoholysis (Ic), step (C) comprises blending the chemolysis product with an organic solvent incompletely miscible with the alcohol used in step (B), and phase separation into the first product phase and the second product phase,
or
(iii) in the case of performance of the chemolysis as alcoholysis (Ia) or hydroalcoholysis (Ic), step (C) comprises:
(C.I) mixing the chemolysis product with an organic solvent miscible with the alcohol used in step (B) to obtain a product mixture, and
(C.II) washing the product mixture with an aqueous washing liquid and phase separation into the first product phase and the second product phase.

5. The method as claimed in claim 2, further comprising:
(D) obtaining the mono- and/or polyols from the first product phase.

6. The method as claimed in claim 2, further comprising:
(E) obtaining the amines from the second product phase.

7. The method as claimed in claim 6, in which
(i) in the case of performance of the chemolysis as alcoholysis (Ia), step (E) comprises a hydrolysis of the carbamates to amines and a distillative removal of alcohol and water, followed by a distillative purification of the amines remaining after the distillative removal,
or
(ii) in the case of performance of the chemolysis as hydrolysis (Ib) or hydroalcoholysis (Ic), step (E) comprises a distillative removal of alcohol and water from the second product phase, followed by a distillative purification of the amines remaining after the distillative removal.

8. The method as claimed in claim 1, in which, in the case of performance of the chemolysis as alcoholysis (Ia) or hydroalcoholysis (Ic), the alcohol used in the chemolysis comprises methanol, ethanol, ethylene glycol, diethylene glycol, propylene glycol, dipropylene glycol, methylglycol, triethylene glycol, glycerol, 2-methylpropane-1,3-diol or a mixture of two or more thereof.

9. The method as claimed in claim 1, in which the element from the fifth, fourteenth or fifteenth group of the Periodic Table of the Elements comprises vanadium, carbon, silicon orand phosphorus.

10. The method as claimed in claim 9, in which
(i) in the case of performance of the chemolysis as alcoholysis (Ia), the salt of the oxoacid comprises an anion comprising orthovanadate, orthophosphate, diphosphate, triphosphate, tetraphosphate, metaphosphate, alkylphosphonate, arylphosphonate, hydrogenorthosilicate, metasilicate, hydrogenmetasilicate, dihydrogenorthosilicate, trihydrogenorthosilicate, alkylsilanolate or arylsilanolate,
(ii) in the case of performance of the chemolysis as hydrolysis (Ib), the salt of the oxoacid comprises an anion comprising orthovanadate, carbonate, orthophosphate, diphosphate, triphosphate, tetraphosphate, metaphosphate, alkylphosphonate, arylphosphonate, hydrogenorthosilicate, metasilicate, hydrogenmetasilicate, dihydrogenorthosilicate, trihydrogenorthosilicate, alkylsilanolate or arylsilanolate,
or
(iii) in the case of performance of the chemolysis as hydroalcoholysis (Ic), the salt of the oxoacid comprises an anion comprising orthovanadate, diphosphate, triphosphate, tetraphosphate, alkylphosphonate, arylphosphonate, hydrogenorthosilicate, metasilicate, hydrogenmetasilicate, dihydrogenorthosilicate, trihydrogenorthosilicate, alkylsilanolate or arylsilanolate.

11. The method as claimed in claim 9, in which the salt of the oxoacid comprises an anion comprising orthovanadate, diphosphate, triphosphate, tetraphosphate, alkylphosphonate, arylphosphonate, hydrogenorthosilicate, metasilicate, hydrogenmetasilicate, dihydrogenorthosilicate, trihydrogenorthosilicate, alkylsilanolate or arylsilanolate.

12. The method as claimed in claim 10, with the proviso that the salt of the oxoacid does not comprise any other anions.

13. The method as claimed in claim 1, in which the isocyanate component comprises phenyl isocyanate, tolylene diisocyanate a di- and/or polyisocyanate of the diphenylmethane series, pentane 1,5-diisocyanate, hexamethylene 1,6-diisocyanate, isophorone diisocyanate, xylylene diisocyanate or a mixture of two or more thereof.

14. The method as claimed in claim 1, in which the mass of the salt of the oxoacid is 0.10% to 20% of the mass of the urethane.

15. The method as claimed in claim 1, in which a phase transfer catalyst is used in addition to the catalyst in the chemolysis.

16. The method as claimed in claim 1 in which the mass ratio of chemolysis reagent to the urethane is in the range from 0.05 to 90.

17. The method as claimed in claim 11, with the proviso that the salt of the oxoacid does not comprise any other anions.

18. The method of claim 1, in which the alcohol component comprises a monool or polyol selected from a polyether monool, a polyether polyol, a polyester polyol, a polyetherester polyol, a polyacrylate polyol, a polyethercarbonate polyol or a mixture of two or more thereof.
